(19) United States
(12) Patent Application Publication
(10) Pub. No.: US 2023/0347393 A1
(43) Pub. Date: Nov. 2, 2023

(54) DEVICE FOR COLLECTING, STORING AND TRANSPORTING FERMENTABLE WASTE

(71) Applicants: Francois-Xavier Postel, Point-Du-Casse (FR); Xavier Hindelang, Mercy-Le Bas (FR)

(72) Inventors: François-Xavier Postel, Pont-Du-Casse (FR); Xavier Hindelang, Mercy-Le-Bas (FR)

(21) Appl. No.: 17/597,497

(22) PCT Filed: Jul. 3, 2020
(86) PCT No.: PCT/FR2020/051183

§ 371 Date: Jan. 7, 2022

(30) Foreign Application Priority Data
Jul. 8, 2019 (FR) 1907626

(51) Int. Cl. B09B 3/35; B09B 3/40; B01D 53/85; B01D 53/04; B65F 1/10; B65F 1/14

(52) U.S. Cl. CPC B09B 3/35; B09B 3/40; B01D 53/85; B01D 53/04; B65F 1/10; B65F 1/14; B01D 2253/102; B01D 2257/90; B01D 2258/05; B65F 2210/1443; B65F 2001/1489; B65F 2210/12

(57) ABSTRACT

A removable device for collecting, storing and transporting fermentable waste comprises, in a direction of flow of the fermentable waste, a feed device for supplying fermentable waste; a hopper for conveying the fermentable waste from the feed device to the grinder; a grinder arranged to grind the fermentable waste and form ground materials; a transfer device for transferring the ground materials to the storage tank; a storage tank for storing the ground materials; a device for emptying the storage tank; and an effluent filtering device connected to the storage tank. The transfer device is under positive pressure in the direction of flow of the fermentable waste, and the effluent filtering device is a solid filtering medium, preferably a carbon filter or a biofilter. The device further comprises a pressure relief valve.

### CROSS-REFERENCE TO RELATED APPLICATIONS

This application is a national phase entry under 35 U.S.C. § 371 of International Patent Application PCT/FR2020/051183, filed Jul. 3, 2020, designating the United States of America and published as International Patent Publication WO 2021/005292 A1 on Jan. 14, 2021, which claims the benefit under Article 8 of the Patent Cooperation Treaty to French Patent Application Serial No. 1907626, filed Jul. 8, 2019.

### TECHNICAL FIELD

The present disclosure relates to a device for collecting, storing and transporting fermentable waste.

### BACKGROUND

Fermentable waste is residues of animal or plant origin composed of biodegradable organic matter. It can be broken down by microorganisms for which it is a source of food. There are several types of fermentable waste: food residues from animal products: meat, milk, cheese, eggshells; food scraps of plant origin such as vegetable peels, fruit peels, used vegetable oils, coffee, tea; green bio-waste or yard waste such as grass clippings, dead leaves, manure, straw; residues consisting of paper and cardboard.

Putrescible waste is remains of animal or plant origin liable to degrade spontaneously and in a very short time, without the addition of microorganisms. It is easily recoverable bio-waste. Not all fermentable waste is necessarily putrescible. However, all putrescible waste is fermentable waste. The difference between putrescible waste and fermentable waste lies in the fact that not all fermentable waste necessarily has the ability to degrade spontaneously. Indeed, cardboard, paper and certain textiles of organic origin require a supply of specific microorganisms in order to decompose.

Fermentable waste is likely to be treated by composting or methanization. Green waste, kitchen scraps and vegetable scraps are examples of fermentable waste.

Composting is an aerobic biological process of converting and enhancing organic matter into a stabilized, hygienic, potting soil-like product, rich in humic and mineral compounds, called compost.

Methanization (or anaerobic digestion) is a natural biological process of degrading anaerobic organic matter, that is to say, in the absence of oxygen. It occurs naturally in certain sediments, marshes, rice fields, landfills, as well as in the digestive tract of certain animals: insects (termites) or vertebrates (ruminants, etc.). Some of the organic matter is broken down into methane, and some is used by methanogenic microorganisms for their growth and reproduction. The decomposition is not complete and leaves the digestate. Methanization is a technique implemented in a digester, also called a biogas reactor or biogas plant, in which the process is accelerated and maintained to produce a usable methane, called biogas, or even biomethane after purification. Thus, fermentable waste, and, in particular, organic waste (or products from solid or liquid energy crops), can provide energy.

There are different shapes and sizes of digesters. In one of the rudimentary techniques used, the digester is in the form of an airtight pit, into which black water, sludge and additional organic compounds are poured to facilitate digestion. Gas forms in the sludge and rises to the surface, mixing the sludge in this process. The digested sludge accumulated at the bottom forms part of the digestate, and can be drained and used as fertilizer.

Prior to composting or methanization of the fermentable material, it is necessary to store and transport the fermentable material to one or more places where the latter can be treated, either by composting or by methanization.

Document LU92556 discloses a fermentable waste storage and transport system provided with a fermentable waste storage device comprising a container for storing the waste, a pump for establishing and maintaining a reduced pressure in the container, a mechanical storage installation for storing the container and the pump, and a transporter for transporting the container and the pump to and from the storage facility. According to the patent document, using a pump to establish and maintain a reduced pressure in the container has the advantage of reducing the quantity of oxygen within the container, and thus of slowing down the decomposition of the waste. The nuisances associated with the decomposition can thus be delayed and the frequency of waste collection can be reduced. However, using the pump to establish a reduced pressure in the container causes the inlet and outlet ports to be suctioned. In order to be able to add the ground fermentable material to the storage tank, which is under a slight depression, it is necessary to stop the pump and to introduce oxygen into the tank to regain atmospheric pressure. The introduced oxygen accelerates the decomposition of the ground materials in the storage tank. Furthermore, it is then necessary to use the pump again to put the storage tank under a slight depression. Either the pump flow rate is high and the effluent filtering means is then ineffective because the gas flow rate passing through it is not nominal but too high, or the pump flow rate is low and it is then necessary to wait a long time, that is to say, of the order of a few hours depending on the volume transferred, in order to be able to start introducing ground fermentable materials into the storage tank again.

Document LU92859 discloses a device for storing and/or transporting organic waste comprising a hermetic tank intended to receive the waste; a vacuum pump fluidly connected to the waste tank and configured to suck the gases present in the tank and to create a vacuum in the tank; and a waste discharge chute, fluidly connected by a pipe to the waste tank. The device further comprises a unit for neutralizing odors emitted by the gases sucked in by the vacuum pump. The document notes that in practice, however, slowing the decomposition of the waste is not particularly useful. Indeed, when the device is transported by a truck and if the truck is used efficiently during a collection round, the truck moves from place to place to collect the waste over a short period of time, typically one or more days, until it is full. However, this waste does not decompose in a few days, even in high heat. Once the tank is full, the truck can then go and empty its contents at an organic waste treatment center. The storage device described in the document also uses the vacuum pump to transfer the waste from a pipe of the device to the interior of the tank. The problems associated with the use of a vacuum pump previously mentioned are still present.

Document LU100173 discloses a device for storing and/or transporting organic waste, comprising a hermetic tank intended to receive the waste; a waste discharge chute, connected by a pipe to the waste tank and means for discharging the gases present in the tank. The device comprises a unit for neutralizing odors emitted by the gases, fluidly connected to the tank by the discharge means and comprising at least one column of aqueous solution, the unit for neutralizing odors being configured to receive the gases from the tank at the bottom of the column(s). The discharge of the waste and its transfer from the supply pipe to the hermetic tank can be carried out by suction or by a worm. The main problem associated with implementing at least one washing column lies in developing the assay of the acidic and basic reagents to be placed in the columns. Indeed, the latter strongly depend on the nature of the waste that will be inserted into the tank, a nature that by definition is unknown before its use. To be effective, the diffusion rate in the washing columns must be slow and constant (bubbling). However, this is not the case when a large influx of gas occurs, for example, when the contents of a garbage can are transferred from the grinder to an almost full tank or when a powerful vacuum pump is used that will not allow the bubbling of the effluent in the washing columns.

### BRIEF SUMMARY

One particular aim of the present disclosure is to remedy all or part of the aforementioned drawbacks.

The present disclosure relates, in particular, to the treatment of putrescible fermentable waste. It can also be applied to the treatment of non-putrescible fermentable waste.

According to a first aspect of the present disclosure, there is proposed a removable device for collecting, storing and transporting fermentable waste comprising, in a direction of flow of the fermentable waste:
a feed device for supplying fermentable waste,
a hopper,
a grinder arranged to grind the fermentable waste and form ground materials,
a transfer device for transferring the ground materials,
a storage tank, preferably airtight, arranged to store the ground materials, preferably under a controlled atmosphere, more preferably anaerobically, and
a tank emptying device arranged to empty the storage tank.

The hopper is arranged to convey the fermentable waste from the feed device to the grinder, the transfer device is arranged to transfer the ground materials from the grinder to the storage tank, and the device further comprises an effluent filtering device (fluidly) connected to the storage tank.

According to the first aspect of the present disclosure, the transfer device can be under positive pressure along the direction of flow of the fermentable waste. The positive pressure transfer device can, for example, be a worm or a peristaltic pump.

The device further comprises a pressure relief valve.

The feed device may comprise manual opening, preferably with a slide valve or a drum.

The feed device may comprise a container lifter, preferably electrically or hydraulically actuated, arranged to empty a rolling bin.

The feed device can be of the gravity type.

The hopper can be provided with a non-stick coating of the polytetrafluoroethylene (PTFE) or polypropylene (PP) type. Polypropylene has a very low coefficient of friction and good resistance to acids and bases.

The transfer device may comprise a gooseneck cylinder, or pipe, which may be connected above the tank.

The transfer device can be under positive pressure along the direction of flow of the fermentable waste. The positive pressure transfer device can, for example, be a worm or a peristaltic pump.

The storage tank can be made of polymer, soft metal or stainless steel.

The storage tank can be covered, at least partially, with a protective and/or reinforcing layer, for example, of the PTFE, stainless steel or epoxy sheet type.

The storage tank may comprise wave-breaking discs arranged vertically.

The storage tank can comprise level sensors.

The storage tank may further comprise heating means arranged to heat the ground materials.

The device according to the first aspect of the present disclosure can further comprise control means configured to control the heating means.

The effluent filtering device can be arranged (fluidly connected) between the storage tank and the effluent discharge device.

The effluent filtering device may be a solid filtering medium, preferably a carbon filter or a biofilter.

The effluent filtering device may comprise washing columns.

The device according to the present disclosure may comprise an odor trap arranged upstream of the hopper, along the direction of flow of the fermentable materials. The odor trap can be formed by a hopper cover provided with a seal. The cover has at least two functions, one for operating safety of the grinder, the second for sealing the hopper. When closed, the cover allows the release of the grinder 106 in complete safety and retains the odors from the residues of waste that has not been sent to the storage tank.

The device according to the present disclosure may comprise a second odor trap arranged between the transfer device and the storage tank. The second odor trap can be formed by a knife gate valve. The second odor trap ensures that the ground materials cannot return to the grinder. This assembly is particularly advantageous when the transfer device has a gooseneck pipe.

The device according to the present disclosure can further comprise a pressure reducer arranged between the storage tank and the effluent filtering device.

The device according to the present disclosure may further comprise a device for discharging effluent generated by the ground materials and/or fermentable waste fluidly connected to the storage tank.

The device according to the present disclosure may further comprise a device for storing effluent fluidly arranged between the storage tank and the effluent discharge device.

The emptying device can be by gravity or suction, for example, by means of a manhole or an agricultural valve.

According to a second aspect of the present disclosure, there is proposed a transporter equipped with a device according to the first aspect of the present disclosure, or one or more of its improvements, from a storage facility in which the device is stored to another storage facility.

The transporter can be a truck. The type of transporter used may depend on the size and shape of the device, and the terrain over which the container is to be transported.

### BRIEF DESCRIPTION OF THE DRAWINGS

Other advantages and particularities of the present disclosure will become apparent on reading the detailed description of implementations and embodiments, which are in no way limiting, with reference to the accompanying drawings, in which:

FIG. 1 schematically shows a first embodiment of a device for treating fermentable waste according to the present disclosure;

FIG. 2 schematically shows a detail of the device shown in FIG. 1;

FIG. 3 schematically shows another detail of the device shown in FIG. 1; and

FIG. 4 schematically shows yet another detail of the device shown in FIG. 1.

### DETAILED DESCRIPTION

Since the embodiments described hereinafter are not limiting in nature, it is possible, in particular, to consider variants of the present disclosure that comprise only a selection of the features that are described, provided that this selection of features is sufficient to confer a technical advantage or to differentiate the present disclosure from the prior art. This selection comprises at least one preferably functional feature without structural details, or with only a portion of the structural details if this portion alone is sufficient to confer a technical advantage or to differentiate embodiments of the present disclosure from the prior art.

In the figures, an element appearing in a plurality of figures retains the same reference.

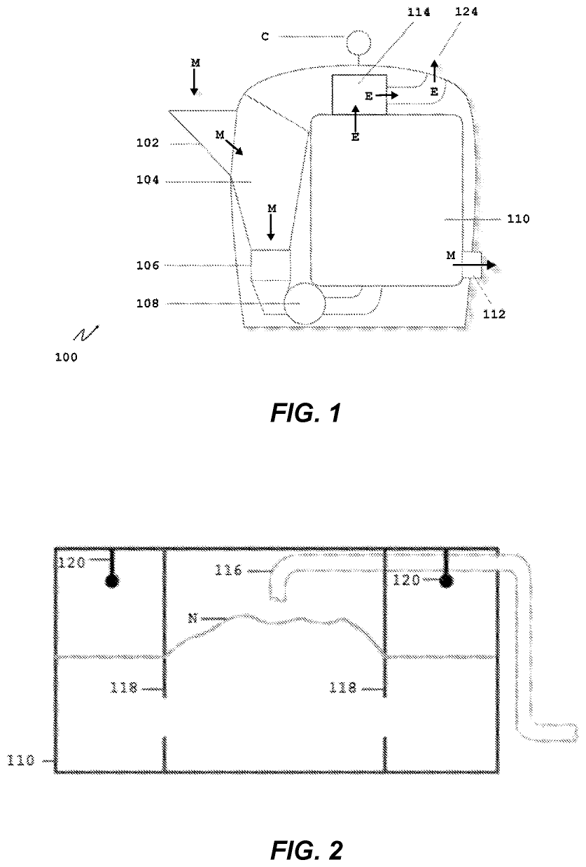

FIG. 1 illustrates an embodiment of a device 100 for collecting, storing and transporting fermentable waste.

The device 100 is removable and movable between a place for collecting waste and a place for unloading the collected waste.

To this end, the device 100 comprises, in a direction of flow M of the fermentable waste:
a feed device 102 for supplying fermentable waste,
a hopper 104,
a grinder 106 designed to grind the fermentable waste and form ground materials,
a transfer device 108 for transferring the ground materials,
a storage tank 110 arranged to store the ground materials, and
a tank emptying device 112 arranged to empty the storage tank 110.

The device 100 is further equipped with a gripping hook C.

The hopper is arranged to convey the fermentable waste from the feed device 102 to the grinder 106.

The transfer device 108 is arranged to transfer the ground materials from the grinder to the storage tank 110.

The device 100 further comprises an effluent filtering device 114 for the effluent (E) that is fluidly connected to the storage tank 110. Gases can flow from the storage tank 110 to the outside of the device 100 through the effluent filtering device 114.

The feed device 102 is of the gravity type.

The feed device 102 may comprise manual opening, preferably with a slide valve or a drum. The feed device may, alternatively or in addition, comprise a container lifter, preferably electrically or hydraulically actuated, arranged to empty a rolling bin.

In the illustrated embodiment, the storage tank is made of polymer, soft metal or stainless steel. The storage tank can be covered, at least partially, with a protective and/or reinforcing layer, for example, of the PTFE (polytetrafluoroethylene), stainless steel or epoxy sheet type.

According to one embodiment shown in FIG. 2, the transfer device 108 may comprise a gooseneck pipe or cylinder 116 connected above the storage tank 110.

Still with reference to FIG. 2, the storage tank 110 comprises wave-breaking discs 118, arranged vertically. These discs, which have the double advantage of optimizing the complete filling of the storage tank 110, limit the risks of overturning during transport. It is thus possible to avoid water hammers.

The storage tank 110 further comprises level sensors 120. They can be placed on either side of the wave breakers 118 and improve the reading of the actual filling level of the tank.

Curve N illustrates a possible spread of the ground materials in the storage tank 110.

With reference to FIG. 1, the hopper 104 may be provided with a non-stick coating, for example, of the PTFE or PP (polypropylene) type. In particular, polypropylene has a very low coefficient of friction and good resistance to acids and bases.

The device 100 can further comprise two odor traps (not shown).

The first odor trap is formed by a cover of the hopper 104 provided with a seal and is thus arranged upstream of the hopper, along the direction of flow of the fermentable materials. The cover has at least two functions, one of safety, the other of sealing the hopper. When closed, it allows the release of the grinder 106 in complete safety and retains the odors from the residues of waste that has not been sent to the storage tank 110.

The second odor trap is formed by a knife gate valve, arranged between the transfer device 108 and the storage tank 110. The knife gate valve ensures that the ground materials cannot return to the grinder 106. This assembly is particularly advantageous when the transfer device has a gooseneck pipe.

Alternatively, the transfer device is under positive pressure along the direction of flow of the fermentable waste. The transfer device 108 can, for example, be a worm, also called Archimedes screw, or a peristaltic pump.

The emptying device 112 operates by gravity or suction, for example, by means of an agricultural valve or a manhole.

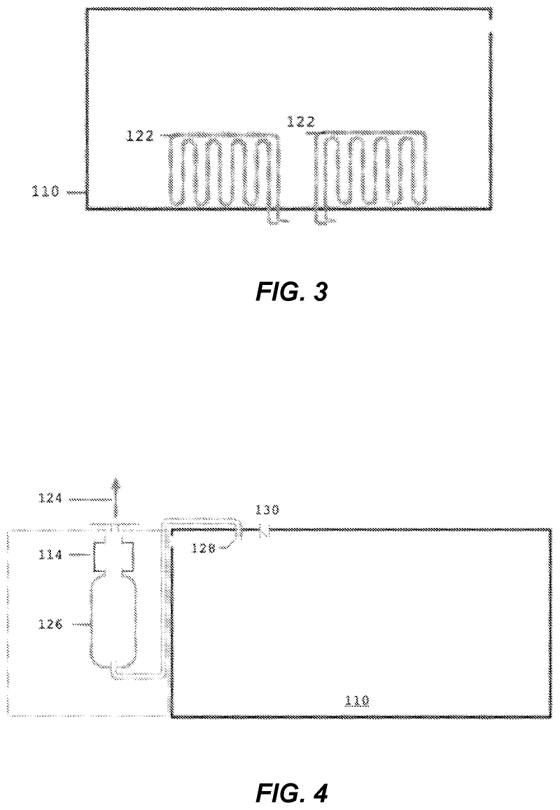

According to one embodiment shown in FIG. 3, the storage tank 110 may further comprise heating means 122 arranged to heat the ground materials.

Thus, once the storage tank 110 is full, it can be heated to a minimum temperature of 70° C. for a minimum period of one hour in order to carry out the phase of hygienization of the fermentable waste. This phase is mandatory for fermentable waste that may contain type C3 animal by-products (SPAN C3). Carrying out this operation in the tank then makes it possible to empty the tank at any biogas plant, regardless of its facilities. Indeed, since the cost of a hygienization facility in a biogas plant is high, few are equipped with one. Furthermore, a biogas plant that uses mainly agricultural products cannot make a hygienization facility profitable.

Control devices, such as computers, can be provided to control the heating means 122.

According to one embodiment shown in FIG. 4, the effluent filtering device 114 is arranged (fluidly connected) between the storage tank 110 and an effluent discharge device 124, generated by the ground materials and/or fermentable waste, of the device 100.

The effluent filtering device 114 is a solid filtering medium, preferably a carbon filter or a biofilter.

The effluent filtering device comprises a pressure relief valve 128, arranged between the storage tank 110 and the effluent filtering device 114, preferably calibrated at 0.1 bar.

In the illustrated example, the storage tank 110 further comprises a safety valve 130 calibrated to 0.3 bar at the interface between the storage tank 110 and the outside of the device 100.

The pressure relief valve 128 and the safety valve 130 are arranged as far as possible from the agricultural valve or from the manhole.

For example, the agricultural valve or the manhole can be arranged on one side, called the front side, of the device 100, while the pressure relief valve 128 and the safety valve 130 are arranged on the other side, called the rear side, of the device 100.

As illustrated in FIG. 4, the device 100 may comprise a pressure reducer 126, arranged between the storage tank 110 and the effluent filtering device 114.

In this case, the pressure relief valve 128 can be arranged between the storage tank 110 and the effluent filtering device 114.

The pressure reducer 126 is chosen carefully and has a volume greater than or equal to the maximum quantity of waste ingestible by the machine in one cycle, for example, 240 L. Thus, it is ensured that the effluent filtering device 114 operates at zero pressure. The effluent filtering device 114 thus exhibits the best filtration rate.

The device 100 may further comprise an effluent storage device, fluidly arranged between the effluent filtering device 114 and the effluent discharge device 124.

As will be readily understood, the present disclosure is not limited to the examples that have just been described, and numerous modifications can be made to these examples without departing from the scope of the invention as defined by the claims. In addition, the various features, forms, variants and embodiments of the present disclosure can be grouped together in various combinations as long as they are not incompatible or mutually exclusive.

1. A removable device for collecting, storing and transporting fermentable waste comprising, in a direction of flow of the fermentable waste:
a feed device for supplying fermentable waste;
a hopper;
a grinder arranged to grind the fermentable waste and form ground materials;
a transfer device for transferring the ground materials;
a storage tank arranged to store the ground materials;
a tank emptying device arranged to empty the storage tank;
an effluent filtering device connected to the storage tank, the effluent filtering device comprising a solid filtering medium; and
a pressure relief valve;
wherein the hopper is arranged to convey the fermentable waste from the feed device to the grinder, the transfer device is arranged to transfer the ground materials from the grinder to the storage tank, and the transfer device is under positive pressure in the direction of flow of the fermentable waste.

2. The device of claim 1, wherein the positive pressure transfer device comprises a worm or a peristaltic pump.

3. The device of claim 2, further comprising an odor trap arranged between the grinder and the storage tank.

4. The device of claim 3, wherein the odor trap comprises a knife gate valve.

5. The device of claim 4, wherein the storage tank comprises wave-breaking discs arranged vertically.

6. The device of claim 5, wherein the storage tank comprises at least one level sensor.

7. The device of claim 6, wherein the storage tank further comprises a heater arranged to heat the ground materials.

8. The device of claim 7, further comprising a controller configured to control the heater.

9. The device of claim 8, further comprising a pressure reducer arranged between the storage tank and the effluent filtering device.

10. The device of claim 9, further comprising a device for storing effluent arranged between the storage tank and the effluent discharge device.

11. A transporter equipped with a device according to claim 1, the transporter configured to transport the device from a storage facility in which the device is stored to another storage facility.

12. The device of claim 1, wherein the solid filtering medium comprises a carbon filter or a biofilter.

13. The device of claim 1, further comprising an odor trap arranged between the grinder and the storage tank.

14. The device of claim 13, wherein the odor trap comprises a knife gate valve.

15. The device of claim 1, wherein the storage tank comprises wave-breaking discs arranged vertically.

16. The device of claim 1, wherein the storage tank comprises at least one level sensor.

17. The device of claim 1, wherein the storage tank further comprises a heater arranged to heat the ground materials.

18. The device of claim 17, further comprising a controller configured to control the heater.

19. The device of claim 1, further comprising a pressure reducer arranged between the storage tank and the effluent filtering device.

20. The device of claim 1, further comprising a device for storing effluent arranged between the storage tank and the effluent discharge device.
